(19) United States
(12) Patent Application Publication
(10) Pub. No.: US 2023/0147835 A1
(43) Pub. Date: May 11, 2023

(54) METHODS OF TREATING SYSTEMIC SCLEROSIS

(71) Applicant: Horizon Therapeutics Ireland DAC, Dublin (IE)

(72) Inventors: Paul PELOSO, Deerfield, IL (US); Farah ALI, Deerfield, IL (US)

(21) Appl. No.: 17/907,589

(22) PCT Filed: Apr. 2, 2021
(86) PCT No.: PCT/US2021/025505

§ 371 Date: Sep. 28, 2022

Related U.S. Application Data
(60) Provisional application No. 63/004,322, filed on Apr. 2, 2020.
(60) Provisional application No. 63/154,203, filed on Feb. 26, 2021.

(51) Int. Cl. A61K 31/196; A61K 45/06; A61P 17/00

(52) U.S. Cl. CPC A61K 31/196; A61K 45/06; A61P 17/00

(57) ABSTRACT

Provided is a method of treating systemic sclerosis in a patient in need thereof, comprising administering to the patient a therapeutically effective amount of 2-(4-methoxy-3-(3-methylphenethoxy)benzamido)-2,3-dihydro-1H-indene-2-carboxylic acid (Compound 1), or a pharmaceutically acceptable salt thereof, for a dosing period of at least about 24 consecutive weeks.

Systemic sclerosis is a rare generalized disease with scleroderma, i.e., skin thickening, as one of the most common symptoms. The disease has two main subsets, diffuse and limited, which are distinguished based on the extent of skin involvement.

Typically, limited scleroderma (also known as CREST syndrome) involves cutaneous manifestations that mainly affect the hands, arms, and face. The limited subtype also typically involves a long previous history of Raynaud's phenomenon, whereas in the diffuse subtype, onset of Raynaud's phenomenon can be simultaneous with other manifestations or might occur later. Both limited and diffuse subtypes may involve internal organs. Typical visceral manifestations of limited systemic sclerosis include isolated pulmonary hypertension, severe bowel involvement, and pulmonary fibrosis. Typical visceral manifestations of diffuse systemic sclerosis include gastro-intestinal disease, renal crisis, lung fibrosis, and vascular involvement including cardiac disease. Diffuse systemic sclerosis typically progresses rapidly and affects a large area of the skin and has more rapid and more severe involvement in one or more internal organs (e.g., kidneys, esophagus, heart, lungs, gastro-intestinal tract). Systemic sclerosis sine scleroderma is a rare disorder in which patients develop vascular and fibrotic damage to internal organs in the absence of cutaneous sclerosis.

Despite the advances that have been made in this field, most existing therapies remain symptomatic treatment only and there remains a need for new therapeutic products useful to treatment of systemic sclerosis and other related diseases or conditions described herein.

Provided is a method of treating systemic sclerosis in a patient in need thereof, comprising:
administering to the patient a therapeutically effective amount of 2-(4-methoxy-3-(3-methylphenethoxy)benzamido)-2,3-dihydro-1H-indene-2-carboxylic acid (Compound 1)

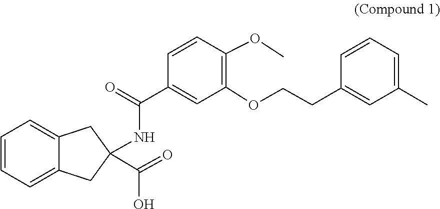 [SMILES: COc1ccc(C(=O)NC2(C(=O)O)Cc3ccccc3C2)cc1OCCc1cccc(C)c1]

or a pharmaceutically acceptable salt thereof, for a dosing period of at least about 24 consecutive weeks.

Also provided is a method for reducing fibrosis in a cell or tissue, the method comprising contacting the cell or tissue with 2-(4-methoxy-3-(3-methylphenethoxy)benzamido)-2,3-dihydro-1H-indene-2-carboxylic acid (Compound 1)

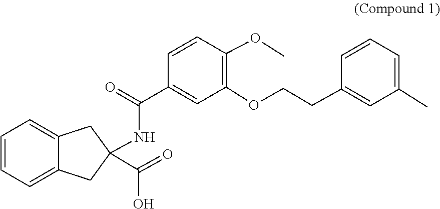 [SMILES: COc1ccc(C(=O)NC2(C(=O)O)Cc3ccccc3C2)cc1OCCc1cccc(C)c1]

or a pharmaceutically acceptable salt thereof, in an amount and for a time sufficient to decrease or inhibit the fibrosis.

These and other aspects of the disclosure will be apparent upon reference to the following detailed description. To this end, various references are set forth herein which describe in more detail certain background information, procedures, compounds, and/or compositions, and are each hereby incorporated by reference in their entirety.

### BRIEF DESCRIPTION OF DRAWINGS

FIG. 1 depicts a histogram demonstrating the median percent change from baseline for various biomarkers after treatment with Compound 1.

### DETAILED DESCRIPTION

As used herein, “Compound 1” means 2-(4-methoxy-3-(3-methylphenethoxy)benzamido)-2,3-dihydro-1H-indene-2-carboxylic acid or 2-[4-methoxy (2-m-tolyl-ethoxy)-benzoylamino]-indane-2-carboxylic acid, including crystalline forms thereof.

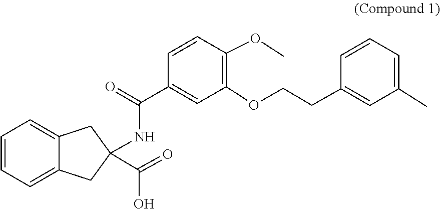 [SMILES: COc1ccc(C(=O)NC2(C(=O)O)Cc3ccccc3C2)cc1OCCc1cccc(C)c1]

See, U.S. Pat. Nos. 9,328,071 and 8,362,073, each of which is hereby incorporated by reference in its entirety. Compound 1 also may be referred to as CZN001, SAR100842, and/or HZN-825.

Compound 1 is a potent antagonist of lysophosphatidic acid receptor 1 (LPAR1). LPAR1 signaling has been implicated in fibrosis and inflammation. Specifically, animal models of fibrosis demonstrate that LPAR1 is involved in fibrosis of skin, lung, kidney, and heart. LPAR1 deletion in mice was found to be protective against dermal and lung fibrosis. LPAR1 antagonism reduced immune cell infiltration into lungs in an experimental model of lung irritation. Serum lysophosphatidic acid (LPA) levels are elevated in patients with systemic sclerosis. Fibroblasts from patients show elevated LPAR1 levels and increased sensitivity to LPAR1 antagonism versus normal fibroblasts.

Compound 1 has been tested in multiple Phase 1 studies and was found to be safe and well tolerated. In a Phase 2 double-blind phase and open label extension, treatment with Compound 1 was found to produce an improvement in Modified Rodnan Skin Score (mRSS) assessing a key feature of systemic sclerosis. See, Khanna, et al. (2007) Curr Rheumatol Rep. 9:151-7, which is incorporated herein by reference for all purposes.

HAQ-DI is 0-3 scale measuring eight domains of daily functioning (dressing, arising, eating, walking, hygiene, reach, grip, and common daily activities. A score of 3 represents severe disability and a score of 0 represents no disability. A minimum clinically important difference for systemic sclerosis has been suggested to be ≥0.14. See, Sultan et al. (2004) Rheumatology 43:472-8, which is incorporated herein by reference for all purposes.

The Physician Global Assessment (MDGA) is an 11-point Likert scale ranging from 0 to 10 (0=excellent to 10=extremely poor) on which the physician rates the subject's overall health over the past week. There is also a 5-point scale (from 1 to 5; 1=much better to 5=much worse) on which the physician rates the subject's overall scleroderma condition compared to the last clinic visit.

The Physician Global Assessment (PTGA) is an 11-point Likert scale ranging from 0 to 10 (0=excellent to 10=extremely poor) on which the subject rates his/her overall health and illness-related pain level over the past week and how much the skin involvement due to scleroderma has interfered with daily activity and how rapidly the skin disease has been progressing over the past month. There is also a 5-point Likert scale (from 1 to 5; 1=much better to 5=much worse) on which the subject rates overall scleroderma skin involvement compared to the last clinic visit.

The Modified Rodnan Skin Score (mRSS) is a validated method for estimating skin thickening. Seventeen different body areas are scored as normal (0), mild thickening (1), moderate thickening (2) and severe thickening (3), with a maximum score of 51.

The Scleroderma Skin Patient-reported Outcome Instrument (SSPRO-18), developed through concept elicitation in patients with diffuse cutaneous systemic sclerosis (dcSSc) and limited cutaneous systemic sclerosis (lcSSC) based on three focus groups, is an 18-item, patient-reported outcome instrument that specifically assesses skin-related quality of life in patients with systemic sclerosis (SSc) and was developed with extensive patient input and according to the FDA patient-reported outcomes guidance [Man et al., 2017]. The SSPRO-18 comprises 4 major conceptual constructs—physical effects, emotional effects, physical limitations, and social effects—and has reproducibility and high internal consistency. This instrument reflects how subjects feel and function from several different health perspectives.

Subjects will be evaluated using the American College of Rheumatology-Composite Response Index in Systemic Sclerosis (CRISS), an outcome measure for dcSSc. The CRISS includes core items that assess change in 2 prominent manifestations of early dcSSc (skin and ILD), functional disability (HAQ-DI) and patient and physician global assessments. In addition, the score captures a clinically meaningful worsening of internal organ involvement requiring treatment.

Forced vital capacity (FVC) is the amount of air that can be forcibly exhaled from your lungs after taking the deepest breath possible, as measured by spirometry.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The phrase “therapeutically effective” is intended to qualify the amount of active ingredients used in the treating a disease or disorder or on the effecting of a clinical endpoint.

The term “therapeutically acceptable” refers to those compounds (or salts, prodrugs, tautomers, zwitterionic forms, etc.) suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease. In certain embodiments, prevention of a disease may involve prevention of attacks of an intermittent nature, as well as prevention of a permanent state of muscle weakness, such as an irreversible state of impairment owing to underlying disease.

The term “patient” is generally synonymous with the term “subject” and includes all mammals including humans Examples of patients include humans, livestock such as cows, goats, sheep, pigs, and rabbits, and companion animals such as dogs, cats, rabbits, and horses. Preferably, the patient is a human.

As used herein, a patient is said to “tolerate” a dose of a compound if administering that dose to that patient does not result in an unacceptable adverse event or an unacceptable combination of adverse events. One of skill in the art will appreciate that tolerance is a subjective measure and that what may be tolerable to one patient may not be tolerable to a different patient. For example, one patient may not be able to tolerate headache, whereas a second patient may find headache tolerable but is not able to tolerate vomiting, whereas for a third patient, either headache alone or vomiting alone is tolerable, but the patient is not able to tolerate the combination of headache and vomiting, even if the severity of each is less than when experienced alone.

As used herein, an “adverse event” is an untoward medical occurrence associated with treatment with a pharmaceutical agent.

The compounds disclosed herein can exist as therapeutically acceptable salts. The present disclosure includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable.

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

When introducing elements of the present disclosure or the embodiment(s) thereof, the articles “a”, “an”, “the” and “said” are intended to mean that there are one or more of the elements. The terms “comprising”, “including” and “having” are inclusive and mean that there may be additional elements other than the listed elements.

The term “and/or” when in a list of two or more items, means that any of the listed items can be employed by itself or in combination with one or more of the listed items. For example, the expression “A and/or B” means either or both of A and B, i.e. A alone, B alone or A and B in combination. The expression “A, B and/or C” is intended to mean A alone, B alone, C alone, A and B in combination, A and C in combination, B and C in combination or A, B, and C in combination.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about” qualifies the numerical values that it modifies, denoting such a value as variable within a margin of error. When no margin of error, such as a standard deviation to a mean value given in a chart or table of data, is recited, the term “about” means that range which would encompass the recited value and the range which would be included by rounding up or down to that figure, considering significant figures.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

Provided is a method of treating systemic sclerosis in a patient in need thereof, comprising:
administering to the patient a therapeutically effective amount of 2-(4-methoxy-3-(3-methylphenethoxy)benzamido)-2,3-dihydro-1H-indene-2-carboxylic acid (Compound 1)

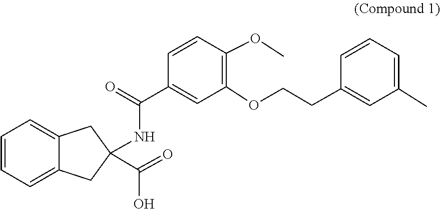 [SMILES: COc1ccc(C(=O)NC2(C(=O)O)Cc3ccccc3C2)cc1OCCc1cccc(C)c1]

or a pharmaceutically acceptable salt thereof, for a dosing period of at least about 24 consecutive weeks.

Also provided is a method for reducing fibrosis in a cell or tissue, the method comprising contacting the cell or tissue with 2-(4-methoxy-3-(3-methylphenethoxy)benzamido)-2,3-dihydro-1H-indene-2-carboxylic acid (Compound 1)

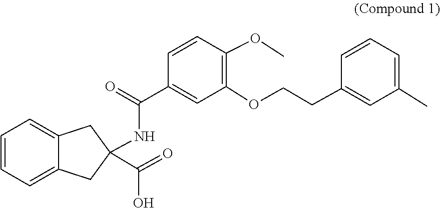 [SMILES: COc1ccc(C(=O)NC2(C(=O)O)Cc3ccccc3C2)cc1OCCc1cccc(C)c1]

or a pharmaceutically acceptable salt thereof, in an amount and for a time sufficient to decrease or inhibit the fibrosis.

In certain embodiments, the systemic sclerosis is chosen from limited cutaneous systemic sclerosis, diffuse cutaneous systemic sclerosis, and systemic sclerosis sine scleroderma.

In certain embodiments, the systemic sclerosis is limited cutaneous systemic sclerosis.

In certain embodiments, the systemic sclerosis is systemic sclerosis sine scleroderma.

In certain embodiments, the systemic sclerosis is diffuse cutaneous systemic sclerosis.

In certain embodiments, the systemic sclerosis is early diffuse cutaneous systemic sclerosis (i.e., wherein it has been less than 5 years from first non-Raynaud's phenomenon sign or symptom in the subject.)

In certain embodiments, the dosing period is at least 36 weeks, such as at least 37, 38, 39, 40, 41, 42, 43, 44, 45, 46, 47, 48, 49, 50, or 51 weeks. In certain embodiments, the dosing period is at least 1 year (52 weeks), such as at least 2, at least 3, or at least 4 years. In certain embodiments, the compound is administered chronically.

In certain embodiments, Compound 1, or a pharmaceutically acceptable salt thereof, is administered orally.

In certain embodiments, 300 mg (on a free acid equivalent weight basis) of Compound 1, or a pharmaceutically acceptable salt thereof, is administered twice daily.

In certain embodiments, 300 mg (on a free acid equivalent weight basis) of Compound 1, or a pharmaceutically acceptable salt thereof, is administered once daily.

In certain embodiments, treatment with Compound 1, or a pharmaceutically acceptable salt thereof, results in an improvement in forced vital capacity (FVC) %.

In certain embodiments, treatment with Compound 1, or a pharmaceutically acceptable salt thereof, results in an improvement in HAQ-DI.

In certain embodiments, treatment with Compound 1, or a pharmaceutically acceptable salt thereof, results in an improvement in MDGA.

In certain embodiments, treatment with Compound 1, or a pharmaceutically acceptable salt thereof, results in an improvement in PTGA.

In certain embodiments, treatment with Compound 1, or a pharmaceutically acceptable salt thereof, results in an improvement in the Physical Effects subscale of the SSPRO-18.

In certain embodiments, treatment with Compound 1, or a pharmaceutically acceptable salt thereof, results in an improvement in the Physical Limitations subscale of the SSPRO-18.

In certain embodiments, treatment with Compound 1, or a pharmaceutically acceptable salt thereof, results in an mRSS decrease of ≥5 points and 25% from Baseline

In certain embodiments, treatment with Compound 1, or a pharmaceutically acceptable salt thereof, results in a CRISS score of greater or equal to 0.60. See, Khanna et al. (2016) Arthritis Rheumatol. 68(2):299-311, which is incorporated by reference in its entirety for all purposes.

In certain embodiments, treatment with Compound 1, or a pharmaceutically acceptable salt thereof, results in a reduction in skin fibrosis as measured by mRSS change of ≥5.

In certain embodiments, treatment with Compound 1, or a pharmaceutically acceptable salt thereof, results in an improvement in HAQ-DI of ≥0.14. In certain embodiments, treatment with Compound 1, or a pharmaceutically acceptable salt thereof, results in an improvement in HAQ-DI of about 0.14 and 0.3.

In certain embodiments, treatment with Compound 1, or a pharmaceutically acceptable salt thereof, results in an improvement in ≥3 of 5 core measures from Baseline: ≥20% in mRSS, ≥20% in HAQ-DI, ≥20% in PTGA, ≥20% in MDGA and ≥5% for FVC % predicted.

In certain embodiments, the method further comprises testing the subject for at least one biomarker. In certain embodiments, the biomarker is selected from Cartilage Oligomeric Matrix Protein (COMP) and thrombospondin-1 (TSP1).

In certain embodiments, the biomarker test is used for early diagnosis or for differential diagnosis, in particular for distinguishing systemic sclerosis from other autoimmune diseases or from rheumatic diseases. In certain embodiments, the biomarker test is used for selecting the dosage and/or frequency of administration of Compound 1, or a pharmaceutically acceptable salt thereof.

In certain embodiments, the method further comprises administering one or more additional therapies.

In certain embodiments, the one or more additional therapies are chosen from an immunosuppressant (e.g., methotrexate, azathioprine, cyclosporine, mycophenolate mofetil, and cyclophosphamide), T-cell-directed therapy (e.g., halofuginone, basiliximab, alemtuzumab, abatacept, rapamycin), B-cell directed therapy (e.g., rituximab), autologous hematopoietic stem cell transplantation, a chemokine ligand receptor antagonist (e.g., an agent that targets the CXCL12/CSCR4 axis (e.g., AMD3100)), a DNA methylation inhibitor (5-azacytidine), a histone deacetylase inhibitor (e.g., trichostatin A), a statin (e.g., atorvastatin, simvastatin, pravastatin), an endothelin receptor antagonist (e.g., bosentan), a phosphodiesterase type V inhibitor (e.g., sildenafil) a prostacyclin analog (e.g., trepostinil), an inhibitor of cytokine synthesis and/or signaling (e.g., imatinib mesylate, rosiglitazone, rapamycin, anti-transforming growth factor beta 1 (anti-TGFβ1) antibody, mycophenolate mofetil, an anti-IL-6 antibody (e.g., tocilizumab)), corticosteroids, nonsteroidal anti-inflammatory drugs, light therapy, and blood pressure medications (e.g., ACE inhibitors).

In certain embodiments, the method further comprises monitoring for one or more adverse events. In certain embodiments, the one or more adverse events is chosen from headache, diarrhea and nausea, fall, orthostatic hypotension, and syncope.

While the disclosed compounds may be administered as the raw chemical, it is also possible to present them as a pharmaceutical formulation. Accordingly, provided herein are pharmaceutical formulations which comprise one or more of certain compounds disclosed herein, or one or more pharmaceutically acceptable salts, esters, prodrugs, amides, or solvates thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art. The pharmaceutical compositions disclosed herein may be manufactured in any manner known in the art, e.g., by means of conventional mixing, dissolving, granulating, dragee-making, levigating, emulsifying, encapsulating, entrapping or compression processes.

In certain embodiments, the Compound of Formula 1, or a pharmaceutically acceptable salt thereof, is formulated using the technology described in one or more of U.S. Pat. Nos. 6,375,986; 7,521,068; 6,592,903; 6,969,529; 7,288,267; 6,431,478; 6,745,962; 6,991,191; 7,244,451; 6,582,285; 6,742,734; 6,976,647; 7,575,184; 7,713,551; 7,695,739; 8,309,136; 7,842,232; 9,345,665; 9,974,746; 9,974,747; and 9,974,748, each of which is incorporated herein by reference in its entirety.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound disclosed herein or a pharmaceutically acceptable salt, ester, amide, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately before use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Certain compounds disclosed herein may be administered topically, that is by non-systemic administration. This includes the application of a compound disclosed herein externally to the epidermis or the buccal cavity and the instillation of such a compound into the ear, eye and nose, such that the compound does not significantly enter the blood stream. In contrast, systemic administration refers to oral, intravenous, intraperitoneal, and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In certain embodiments, the active ingredient may comprise as much as 10% w/w. In other embodiments, it may comprise less than 5% w/w. In certain embodiments, the active ingredient may comprise from 2% w/w to 5% w/w. In other embodiments, it may comprise from 0.1% to 1% w/w of the formulation.

For administration by inhalation, compounds may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds may be a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

In addition to the ingredients particularly mentioned above, the formulations described above may include other agents conventional in the art having regard to the type of formulation in question, for example those suitable for oral administration may include flavoring agents.

In certain embodiments, Compound 1, or a pharmaceutically acceptable salt thereof, may be administered at a dose of from about 10 to 1000 mg per day, optionally in divided doses. (Amounts described herein are provided on a free acid equivalent weight basis.)

In certain embodiments, the subject may receive a dose of between 100 and 1000 mg once daily. In certain embodiments, the dose is between about 250 and 750 mg once or twice daily. In certain embodiments, the dose is 300 mg once daily. In certain embodiments, the dose is 300 mg twice daily. In certain embodiments, the dose is 600 mg once daily.

In certain embodiments, the subject may receive a dose of between 100 and 500 mg twice daily. In certain embodiments, the dose is 100 mg twice daily. In certain embodiments, the dose is 200 mg twice daily. In certain embodiments, the dose is 300 mg twice daily.

The amount of active ingredient that may be combined with the carrier materials to produce a single dosage form will vary depending upon the host treated and the mode of administration.

The compounds can be administered in various modes, e.g., orally, topically, or by injection. The precise amount of compound administered to a patient will be the responsibility of the attendant physician. In certain embodiments, the specific dose level for any patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In any case, multiple therapeutic agents (at least one of which is a compound disclosed herein) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few min to four weeks.

Examples of embodiments of the present disclosure are provided in the following examples. The following examples are presented only by way of illustration and to assist one of ordinary skill in using the disclosure. The examples are not intended in any way to otherwise limit the scope of the disclosure.

### EXAMPLES

### Example 1

Film-coated tablets containing 10 mg, 100 mg, 150 mg or 200 mg of Compound 1 and the following excipients mannitol, microcrystalline cellulose, crospovidone, hypromellose, magnesium stearate, povidone, macrogol, titanium dioxide, sodium laurylsulfate, and docusate sodium were prepared.

### Example 2

The activity of Compound 1, a selective LPAR1 receptor antagonist, was evaluated in dermal fibroblasts from patients with systemic sclerosis and in several models of skin, kidney and heart fibrosis.

Two models of skin fibrosis were used to evaluate the effect of Compound 1 in comparison with imatinib mesylate, used as a reference. In the model of bleomycin-induced skin fibrosis, using a therapeutic protocol, Compound 1 at 50 mg/kg/day was able to reverse dermal thickness, myofibroblast differentiation and collagen content in mouse skin. The effect on these markers was comparable with the effects of imatinib. In tight skin (Tsk-1) mice which are heterozygous for a mutation in the fibrillin gene leading to overproduction of matrix proteins in the absence of inflammatory infiltrates, Compound 1 at 30 mg/kg/day was also able to inhibit the progression of skin fibrosis at a similar level than imatinib.

In addition to its effect on skin fibrosis, Compound 1 also improved kidney function in models of hypertension-induced or nephrotoxicity-induced renal failure, showed beneficial effect on cardiac function and structure in different models of hypertension or diabetes-related cardiac hypertrophy, fibrosis and heart failure. In parallel, Compound 1 showed moderate but significant antithrombotic activity in acute models of coagulation and arterial thrombosis. All these pathological events can be observed in SSc patients at different levels and further support the use of Compound 1 in these patients.

Finally, Compound 1 has a significant inhibitory effect on the accumulation of leukocytes and total inflammatory cells in broncho-alveolar lavage of presensitized mice treated with ovalbumin.

In conclusion, Compound 1 inhibits fibrosis in different organs (skin, kidney, and heart). It improves cardiac systolic and diastolic function and has positive effect on arterial stiffness at mostly unchanged blood pressure and demonstrated antithrombotic activity in two models of thrombosis in rats and in one model in mouse. In addition, Compound 1 reduces inflammation on a model of lung injury exhibiting a predominant Th2-type response.

### Example 3

An 8-week double-blind, randomized, placebo-controlled study followed by a 16-week open-label extension with Compound 1 was performed in patients with early dcSSc who had a baseline modified Rodnan skin thickness score (mRSS) of at least 15. The primary end point was safety during the double-blind phase of the trial. Exploratory end points included the identification of an LPA-induced gene signature in patients' skin.

Seventeen of 32 patients were randomly assigned to receive placebo and 15 to receive Compound 1. Thirty patients participated in the open-label extension study. The most frequent adverse events reported for Compound 1 during the blinded phase were headache, diarrhea, nausea, and falling, and the safety profile was acceptable during the open-label extension. At week 8, the reduction in MRSS was numerically greater in the Compound 1 group than in the placebo group (mean±SD change −3.57±4.18 versus −2.76±4.85; treatment effect −1.2 [95% confidence interval −4.37, 2.02]). A greater reduction of LPA-related genes was observed in skin samples from the Compound 1 group at week 8, indicating LPA₁ target engagement.

Clinical outcomes from the 16-week open label extension study are provided below.

| | Median Decrease in mRSS | Responder Rate |
|---|---|---|
| 24-weeks of continuous | −7.5 | 78.6% |
| treatment | | |
| Subjects switched from | −7.0 | 69.2% |
| placebo to Compound 1 | | |

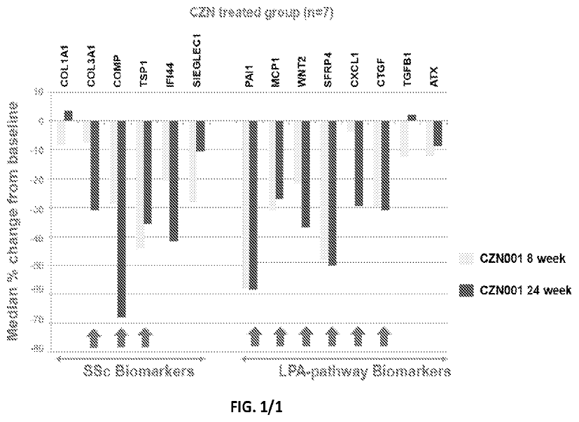

Biomarker analysis of skin biopsies showed reductions in LPA-related genes as shown in FIG. 1. A numerical improvement versus baseline of some disease biomarkers (COMP and TSP1) was observed in the initial Compound 1 group when the treatment duration was extended up to 24 weeks.

Compound 1, a selective orally available LPA1 receptor antagonist, was well tolerated in patients with dcSSc. The MRSS improved during the study although the difference was not significant, and additional gene signature analysis suggested target engagement. In addition, there was clinically significant improvement in HAQ-DI (assesses functional disability) at week 24.

### Example 4

A 52-week double-blind, randomized, placebo-controlled study followed by a 52-week extension with Compound 1 will be performed in patients with early dcSSc who had a baseline modified Rodnan skin thickness score (MRSS) of at least 15 and a forced vital capacity (FVC) of at least 45%. The overall objective is to investigate the efficacy, safety and tolerability of 2 dose regimens of Compound 1, administered once daily (QD) or twice daily (BID) for 52 weeks in the treatment of subjects with dcSSc. The primary objective is to demonstrate the efficacy of 1 or 2 dose regimens of HZN-825 versus placebo in subjects with dcSSc, as determined by a comparison of change in FVC % predicted after 52 weeks of treatment. Additional endpoints as defined above will also be captured and evaluated (e.g., HAQ-DI, SS-PRO, ACR-CRISS, etc.).

The various embodiments described above can be combined to provide further embodiments. All the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary, to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1-14. (canceled)

15. A method of treating systemic sclerosis in a patient in need thereof, comprising administering to the patient a therapeutically effective daily dose of 2-(4-methoxy-3-(3-methylphenethoxy)benzamido)-2,3-dihydro-1H-indene-2-carboxylic acid (Compound 1) 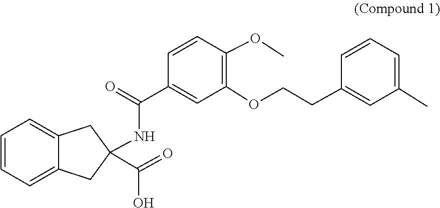 [SMILES: COc1ccc(C(=O)NC2(C(=O)O)Cc3ccccc3C2)cc1OCCc1cccc(C)c1]
or a pharmaceutically acceptable salt thereof, for a dosing period of at least about 24 consecutive weeks.

16. The method of claim 15, wherein the systemic sclerosis is chosen from limited cutaneous systemic sclerosis, diffuse cutaneous systemic sclerosis, and systemic sclerosis sine scleroderma.

17. The method of claim 16, wherein the systemic sclerosis is diffuse cutaneous systemic sclerosis.

18. The method of claim 17, wherein the systemic sclerosis is early diffuse cutaneous systemic sclerosis.

19. The method of claim 15, wherein the systemic sclerosis is limited cutaneous systemic sclerosis.

20. The method of claim 17, wherein the dosing period is at least 36 weeks.

21. The method of claim 17, wherein the dosing period is at least 52 weeks.

22. The method of claim 21, wherein Compound 1, or a pharmaceutically acceptable salt thereof, is administered orally.

23. The method of claim 22, wherein the therapeutically effective daily dose of Compound 1, or a pharmaceutically acceptable salt thereof, is equivalent to 300 mg of Compound 1 administered once daily.

24. The method of claim 23, wherein said treatment with Compound 1, or a pharmaceutically acceptable salt thereof, results in an American College of Rheumatology-Composite Response Index in Systemic Sclerosis (CRISS) score of greater or equal to 0.60.

25. The method of claim 23, wherein said treatment with Compound 1, or a pharmaceutically acceptable salt thereof, results in a reduction in skin fibrosis as measured by Modified Rodnan Skin Score (mRSS) change of ≥5.

26. The method of claim 23, wherein treatment with Compound 1, or a pharmaceutically acceptable salt thereof, results in an improvement in HAQ-DI of ≥0.14.

27. The method of claim 23, wherein said treatment with Compound 1, or a pharmaceutically acceptable salt thereof, results in an improvement in forced vital capacity (FVC) as measured spirometry.

28. The method of claim 23, further comprising administering one or more additional therapies.

29. The method of claim 28, wherein the one or more additional therapies are chosen from an immunosuppressant, T-cell-directed, B-cell directed therapy, autologous hematopoietic stem cell transplantation, a chemokine ligand receptor antagonist, a DNA methylation inhibitor, a histone deacetylase inhibitor, a statin, an endothelin receptor antagonist, a phosphodiesterase type V inhibitor, a prostacyclin analog, an inhibitor of cytokine synthesis and/or signaling, corticosteroids, nonsteroidal anti-inflammatory drugs, light therapy, and blood pressure medications.

30. The method of claim 22, wherein the therapeutically effective daily dose of Compound 1, or a pharmaceutically acceptable salt thereof, is equivalent to 300 mg of Compound 1 administered twice daily.

31. The method of claim 30, wherein said treatment with Compound 1, or a pharmaceutically acceptable salt thereof, results in an American College of Rheumatology-Composite Response Index in Systemic Sclerosis (CRISS) score of greater or equal to 0.60.

32. The method of claim 30, wherein said treatment with Compound 1, or a pharmaceutically acceptable salt thereof, results in a reduction in skin fibrosis as measured by Modified Rodnan Skin Score (mRSS) mRSS change of ≥5.

33. The method of claim 30, wherein treatment with Compound 1, or a pharmaceutically acceptable salt thereof, results in an improvement in HAQ-DI of ≥0.14.

34. The method of claim 30, wherein said treatment with Compound 1, or a pharmaceutically acceptable salt thereof, results in an improvement in forced vital capacity (FVC) as measured spirometry.

35. The method of claim 30, further comprising administering one or more additional therapies.

36. The method of claim 35, wherein the one or more additional therapies are chosen from an immunosuppressant, T-cell-directed, B-cell directed therapy, autologous hematopoietic stem cell transplantation, a chemokine ligand receptor antagonist, a DNA methylation inhibitor, a histone deacetylase inhibitor, a statin, an endothelin receptor antagonist, a phosphodiesterase type V inhibitor, a prostacyclin analog, an inhibitor of cytokine synthesis and/or signaling, corticosteroids, nonsteroidal anti-inflammatory drugs, light therapy, and blood pressure medications.
